(19) United States
(12) Patent Application Publication
(10) Pub. No.: US 2021/0161555 A1
(43) Pub. Date: Jun. 3, 2021

(54) Endoscopic Cannulas and Related Methods

(71) Applicant: CooperSurgical, Inc., Trumbull, CT (US)

(72) Inventors: Thomas William Winegar, Trumbull, CT (US); Rosemary Michelle Garofalo, North Haven, CT (US)

(21) Appl. No.: 17/082,436

(22) Filed: Oct. 28, 2020

Related U.S. Application Data
(60) Provisional application No. 62/942,864, filed on Dec. 3, 2019.

(51) Int. Cl. A61B 17/34; A61B 17/42; A61B 1/05; A61B 1/005

(52) U.S. Cl. CPC A61B 17/3421; A61B 17/42; A61B 2017/00867; A61B 1/0058; A61B 2017/4225; A61B 1/05

(57) ABSTRACT

An endoscopic cannula includes an elongate shaft having a distal end portion sized for insertion in a body cavity and a camera secured to the distal end portion of the elongate shaft. The camera is configured for viewing the body cavity. The distal end portion of the elongate shaft is adjustable between a straight configuration and a bent configuration to examine the body cavity.

### CROSS REFERENCE TO RELATED APPLICATION

This application claims priority to U.S. Provisional Patent Application No. 62/942,864, filed on Dec. 3, 2019, pursuant to 35 USC § 119. The entire content of this provisional application is herein incorporated by reference in its entirety.

### TECHNICAL FIELD

This disclosure relates to adjustable endoscopic cannulas and related methods of steering the adjustable endoscopic cannulas to examine a uterus of a patient.

### BACKGROUND

A hysteroscope is an endoscope that is designed for examining a uterus (e.g., a uterine cavity) of a patient. A hysteroscope typically includes a proximal portion that remains external to the body of a patient during use and a distal portion that is inserted into the uterus of a patient. The distal portion may include a tip that is sized to be inserted through the cervix and into the uterus to view and/or perform a surgery on the uterus, while the proximal portion provides features for manipulating the distal portion. Images captured at the tip of the distal portion can be viewed by a physician to examine the uterine cavity. Once examination has concluded, the distal portion of the hysteroscope is withdrawn from the uterus through the cervix of a patient.

### SUMMARY

In general, this disclosure relates to endoscopic devices and related methods. Such endoscopic devices can be used for viewing and/or performing a surgery on a body cavity of a patient, e.g., a uterus.

In one aspect, an endoscopic cannula includes an endoscopic cannula, including an elongate shaft having a distal end portion sized for insertion in a body cavity, and a camera secured to the distal end portion of the elongate shaft for viewing the body cavity, wherein the distal end portion of the elongate shaft is adjustable between a straight configuration and a bent configuration to examine the body cavity.

Embodiments may include one or more of the following features.

In some embodiments, the distal end portion of the elongate shaft includes one or more relief cuts that generate the bent configuration.

In certain embodiments, the elongate shaft is made of a flexible material.

In some embodiments, a natural or trained shape of the distal end portion of the elongate shaft provides the bent configuration.

In certain embodiments, the endoscopic cannula further includes a malleable metal rod disposed within the elongate shaft and configured to adjust the distal end portion of the elongate shaft into the straight configuration or the bent configuration.

In some embodiments, a lumen of the elongate shaft is configured to allow passage of an operative instrument from a proximal end of the elongate shaft through the distal end portion of the elongate shaft.

In certain embodiments, the elongate shaft is made of a memory alloy material.

In some embodiments, the endoscopic cannula further includes a rigid sheath defining an interior region for receiving the elongate shaft.

In certain embodiments, the rigid sheath is slidable along the elongate shaft to adjust the elongate shaft between the straight configuration and the bent configuration.

In some embodiments, the distal end portion of the elongate shaft transitions between the straight and bent configurations in response to a temperature change of the memory alloy material.

In another aspect, an endoscope includes an elongate shaft having a distal end portion sized for insertion in a body cavity, a camera secured to the distal end portion of the elongate shaft for viewing the body cavity, and a handle extending from a proximal end of the elongate shaft, wherein the distal end portion of the elongate shaft is adjustable between a straight configuration and a bent configuration to examine the body cavity.

In another aspect, a method of using an endoscope includes inserting an elongate shaft of the endoscope into a body cavity of a patient, adjusting a distal end portion of the elongate shaft from a straight configuration to a bent configuration, and acquiring an image of the body cavity using a camera secured to the distal end portion of the elongate shaft.

Embodiments may include one or more of the following features.

In certain embodiments, the method further includes bending the distal end portion of the elongate shaft at one or more relief cuts of the elongate shaft.

In some embodiments, the elongate shaft is made of a flexible material.

In certain embodiments, the method further includes providing the bent configuration with a natural or trained shape at the distal end portion of the elongate shaft.

In some embodiments, the method further includes manipulating a malleable metal rod within a lumen of the elongate shaft to adjust the distal end portion of the elongate shaft into the straight configuration or the bent configuration.

In certain embodiments, the method further includes inserting an operative instrument into a proximal end of the elongate shaft, through a lumen, and through the distal end portion of the elongate shaft.

In some embodiments, the method further includes manipulating the operative instrument to perform a surgery in the body cavity.

In certain embodiments, the elongate shaft is made of a memory alloy material.

In some embodiments, the method further includes sliding a rigid sheath over the elongate shaft to adjust the distal end portion of the elongate shaft into the straight configuration or the bent configuration.

In certain embodiments, the method further includes changing a temperature of the memory alloy material to adjust the distal end portion of the elongate shaft into the straight configuration or the bent configuration.

Embodiments may provide one or more of the following advantages. In some embodiments, the endoscopic devices include steerable cannulas. Such steerable cannulas can allow a user (e.g., a clinician) to manipulate a distal end of the cannula to observe intrauterine anatomy of a patient without manipulating the entire cannula body to achieve visualization of a desired area. Thus, the steerable cannulas can prevent and/or minimize the pain and/or discomfort of the patient that is typically caused by manipulating the entire cannula body to achieve visualization of a desired area. Furthermore, the steerable cannulas can facilitate cannula and camera placement by the user during visualization while advantageously reducing the need for aggressive manipulation of the endoscope during visualization of intrauterine anatomy.

In some implementations, the endoscopic devices include disposable cannulas that eliminate the need for sterilization of the endoscope between patients. Thus, the disposable cannulas may reduce the time required to prepare and set up an endoscope in between patients. Furthermore, the single-use cannulas may be less costly to manufacture, purchase, and/or maintain than non-disposable cannulas.

Other aspects, features, and advantages will be apparent from the description, the drawings, and the claims.

### DESCRIPTION OF DRAWINGS

FIG. 1 is a perspective view of an endoscopic device that can be used to examine a body cavity of a patient.

FIG. 2 is a side view of the endoscopic device of FIG. 1.

FIG. 3 is a top view of the endoscopic device of FIG. 1.

FIG. 4 is a perspective view of a distal end of the endoscopic device of FIG. 1.

FIG. 5 is a perspective cross-sectional view of a connection hub and a handle of the endoscopic device of FIG. 2.

FIG. 6 is a perspective view of the connection hub and the handle of FIG. 5.

FIG. 7 is a perspective view of the connection hub of FIG. 5, with the handle omitted.

FIG. 8 is a perspective cross-sectional view of the handle of FIG. 5.

FIG. 9 is a front view of a display of the endoscopic device of FIG. 2.

FIG. 10 is a rear perspective view of the display of FIG. 9.

FIG. 11 is a rear perspective view of electronics within the display of FIG. 9.

FIG. 12 is a perspective view of the display of FIG. 9, mated with a docking station.

FIG. 13 is a front perspective view of the docking station of FIG. 12.

FIG. 14 is a rear perspective view of the docking station of FIG. 12.

FIG. 15 is a perspective view of an endoscopic cannula including relief cuts in a straight configuration.

FIG. 16 is a perspective view of an endoscopic cannula including relief cuts in a bent configuration.

FIG. 17 is a perspective view of an endoscopic cannula including relief cuts and an operative channel in a straight configuration.

FIG. 18 is a perspective view of an endoscopic cannula including relief cuts and an operative channel in a bent configuration.

FIG. 19 is a perspective view of an endoscopic cannula having an elongate shaft made out of a memory alloy material. The endoscopic cannula is shown in a straight configuration.

FIG. 20 is a perspective view of an endoscopic cannula having an elongate shaft made out of a memory alloy material. The endoscopic cannula is shown in a bent configuration.

FIG. 21 is a perspective view of an endoscopic cannula having an elongate shaft made out of a bendable plastic. The endoscopic cannula is shown in a straight configuration.

FIG. 22 is a perspective view of an endoscopic cannula having an elongate shaft made out of a bendable plastic. The endoscopic cannula is shown in a bent configuration.

FIG. 23 is a side cross-sectional view of an endoscopic cannula including a flexible rod in a straight configuration.

FIG. 24 is a rear cross-sectional view of an endoscopic cannula including a malleable metal rod.

FIG. 25 is a perspective cross-sectional view of an endoscopic cannula including a flexible rod in a bent configuration.

FIG. 26 illustrates the endoscopic cannula of FIGS. 15-16 within a uterine cavity during an endoscopic procedure.

FIG. 27 illustrates the endoscopic cannula of FIGS. 17-18 within a uterine cavity during an endoscopic procedure.

FIG. 28 illustrates the endoscopic cannula of FIGS. 19-20 within a uterine cavity during an endoscopic procedure.

FIG. 29 illustrates the endoscopic cannula of FIGS. 21-22 within a uterine cavity during an endoscopic procedure.

FIG. 30 illustrates the endoscopic cannula of FIGS. 23-25 within a uterine cavity during an endoscopic procedure.

### DETAILED DESCRIPTION

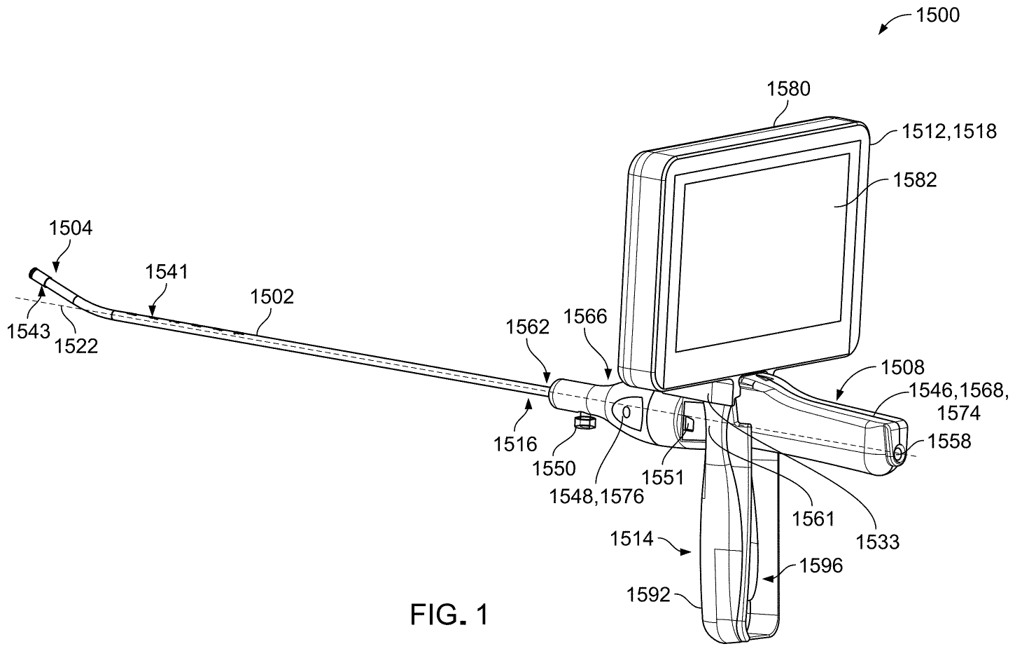

FIGS. 1-3 illustrates an endoscopic device 1500 (e.g., a hysteroscope) that can be used to examine a body cavity of a patient (e.g., a uterine cavity). The endoscopic device 1500 includes a cannula 1502 that is formed to be inserted into the uterus (e.g., through the vaginal canal and cervix of the patient), an imaging system 1504 located at a distal tip 1506 of the cannula 1502 for imaging the uterus, and a connection hub 1508 attached to a proximal end region 1510 of the cannula 1502. The endoscopic device 1500 further includes a display 1512 for viewing images acquired by the imaging system 1504 and a handle 1514 that extends from the display 1512. The cannula 1502, the imaging system 1504, and the connection hub 1508 together form a single-use portion 1516 of the endoscopic device 1500 that is designed to be disposed of following an examination of a uterus of a single patient. The single-use portion 1516 can be provided in a sealed, sterile package that can be stored until a time of use. The display 1512 and the handle 1514 together form a reusable portion 1518 of the endoscopic device 1500 that is designed to be attached to and detached from several single-use portions 1516 to respectively examine multiple uteruses of patients. The reusable portion 1518 is sterilized (e.g., cleaned and disinfected) following examination of each uterus of a patient (e.g., prior to examining a next uterus of another patient). Referring to FIGS. 1-4, the cannula 1502 is an elongate, generally tubular member that is sized to pass through a cervix into a uterus. The cannula 1502 includes a shaft 1520 and a cap 1526 that secures the imaging system 1504 to the distal tip 1506 of the shaft 1520. The shaft 1520 includes a major portion 1530 with a central axis that defines a primary axis 1522 of the cannula 1502, the distal tip 1506, and a distal bend 1524 that connects the major portion 1530 to the distal tip 1506. The shaft 1520 defines a lumen 1528 that houses one or more electrical cables of the imaging system 1504, that allows for passage of fluids between the distal tip 1506 and the connection hub 1508, and that allows for passage of a working tool extending distally from connection hub 1508. The shaft 1520 further defines a sidewall opening along a proximal end region 1510 through which fluid can be delivered to the lumen 1528 or withdrawn (e.g., suctioned) from the lumen 1528.

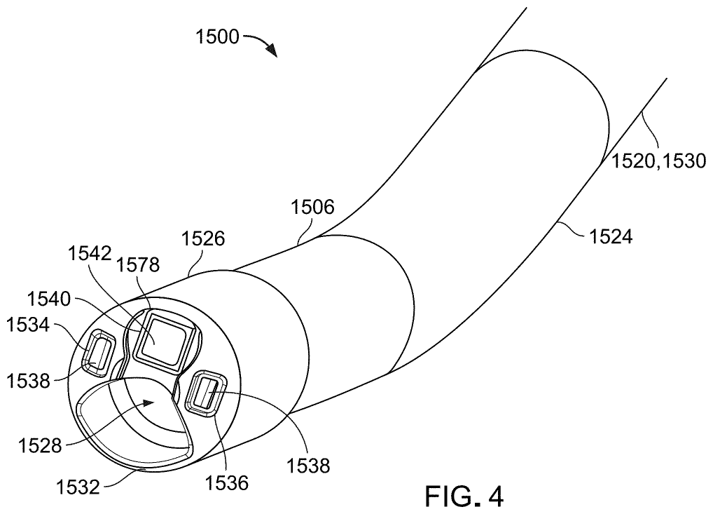

The cap 1526 of the cannula 1502 is secured to the distal tip 1506 of the shaft 1520 and defines multiple openings, as shown in FIG. 4. The openings include a luminal opening 1532 (e.g., a forward facing fluid port) through which fluids and uterine tissue (e.g., endometrial tissue) can enter and exit the lumen 1528 of the shaft 1520, two lateral openings 1534, 1536 in which light emitting diodes (LEDs) 1538 of the imaging system 1504 are disposed, and a recessed opening 1540 in which a camera 1542 of the imaging system 1504 is disposed. An overhanging edge 1578 of the cap 1526 acts as a lens hood that shields light from directly impinging on the LEDs 1538 and from entering an aperture of the camera 1542.

The luminal opening 1532 allows fluid (e.g., a saline solution, a hypotonic solution, or an isotonic fluid) to exit the distal tip 1506 to flow into the uterus and to push tissue or other particulate matter away from the camera 1542 so as to improve a quality of images acquired by the camera 1542. For example, the luminal opening 1532 can be useful in clearing away tissue debris that may collect on the distal tip 1506 and otherwise impair imaging due to an overly bright appearance of the debris as light reflects from the debris. In some cases, the luminal opening 1532 can also facilitate insertion of the cannula 1502, as fluid exiting the luminal opening 1532 may lubricate and partially distend tissues surrounding the distal tip 1506. In this manner, the luminal opening 1532 can reduce a risk of accidental damage to the vaginal cavity, to the cervix, or to the uterus during insertion of the cannula 1502 into the patient. The luminal opening 1532 is sized to permit passage of a 5 French biopsy tool. For example, the luminal opening 1532 typically has a cross-sectional area of about 0.03 cm² to about 0.05 cm² and is about 50% to about 80% of a cross-sectional area of the lumen 1528, itself.

The cannula 1502 typically has a total length (e.g., as measured along the primary axis 1522) of about 30.0 cm to about 34.0 cm (e.g., about 32.0 cm). The proximal end region 1510 of the cannula 1502 (e.g., the portion of the cannula 1502 that is disposed within the connection hub 1508) typically has a length of about 4.0 cm to about 4.6 cm (e.g., about 4.3 cm), such that a remaining portion of the cannula 1502 extends distally from the connection hub 1508 and is therefore exposed for insertion into the patient. The distal bend 1524 typically has a radius of about 2.5 cm to about 7.5 cm (e.g., about 5.0 cm). The shaft 1520 typically has a wall thickness of about 0.03 cm to about 0.05 cm (e.g., about 0.04 cm) and an inner diameter (e.g., a luminal diameter) of about 0.34 cm to about 0.36 cm (e.g., about 0.35 cm).

The shaft 1520 is typically made of one or more materials that are flexible enough to allow the cannula 1502 to bend by a small amount to be appropriately placed within the patient as desired, yet stiff enough to permit easy insertion into the vaginal canal. Example materials from which the shaft 1520 is typically made include nylon, polysulfone, and polyether ether ketone (PEEK). The cannula 1502 is typically manufactured primarily via extrusion and via secondary processes that may include one or more of punching, laser cutting, forming, and/or printing. The cap 1526 is typically made of one or more materials including liquid crystal polymer (LCP) and is typically secured to the distal tip 1506 of the shaft 1520 via adhesive. The cannula 1502 further includes ruled markings 1541 that indicate distances from a distal end 1543 of the cannula 1502. The ruled markings 1541 can be viewed by a user during a laparoscopic procedure to determine a depth to which the cannula 1502 has been inserted into the patient. The ruled markings 1541 may be provided in metric units or English units or provided as a dimensionless scale.

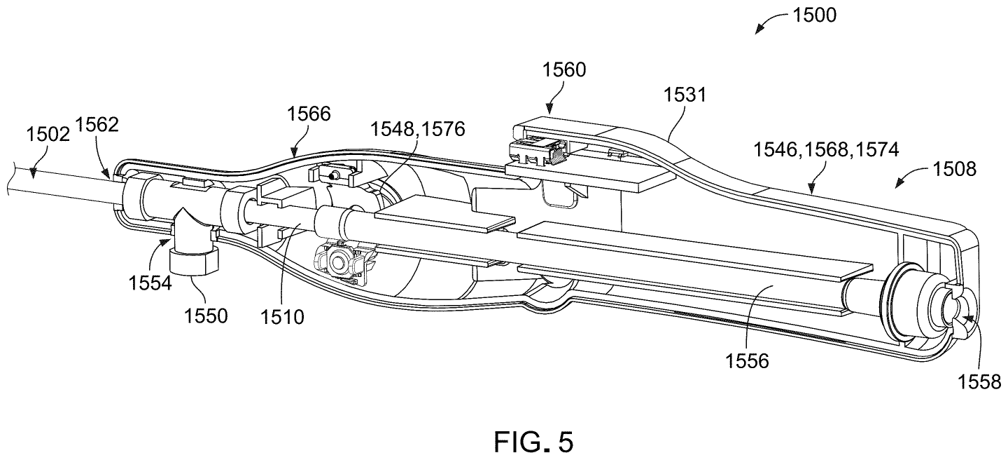

Referring to FIGS. 5-8, the connection hub 1508 surrounds the proximal end region 1510 of the cannula 1502 and serves as a mounting piece for the reusable display 1512. The connection hub 1508 also provides several features for fluid and electrical communication between the proximal end region 1510 of the cannula 1502 and the distal tip 1506 of the cannula 1502. For example, the connection hub 1508 includes a housing 1546, a camera actuator 1548 (e.g., providing two opposite push buttons 1576), a fluid port 1550 located adjacent the proximal end region 1510 of the cannula 1502, an entry port disposed at a proximal opening 1558 of the housing 1546, and a straight operative conduit 1556 that extends from the proximal end region 1510 of the cannula 1502 to the entry port.

The housing 1546 is generally axially aligned with the primary axis 1522 of the cannula 1502 and has a generally curved profile that is laterally symmetric. The housing 1546 defines a distal opening 1562 through which the cannula 1502 passes, an opening 1554 to which the fluid port is secured, the proximal opening 1558, and a horizontally oriented upper connection port 1560 (e.g., a micro HDMI port or another type of port) to which the display 1512 or a display cable can be connected. In this regard, the connection hub 1508 also includes electrical components that communicate the camera actuator 1548 with the connection port 1560. The connection port 1560 defines opposite, elongate flanges 1531 that can be engaged with the display 1512 to secure the display 1512 to the connection hub 1508. The housing 1546 further defines additional internal wall features (e.g., flanges, openings, brackets, tabs, channels etc.) that properly position the fluid port 1550, the camera actuator 1548, the connection port 1560, and the entry port 1552.

A distal portion 1566 of the housing 1546 provides fluid communication between the distal tip 106 of the cannula 1502) and the fluid port 1550 and provides fluid communication between the distal tip 106 and the operative conduit 1556 (e.g., for further fluid communication to the entry port). The distal portion 1566 of the housing 1546 further provides electrical communication between the distal tip 106 of the cannula 102 (and the camera actuator 1548, and between the distal tip 106 and the display 1512 (e.g., via the connection port 1560).

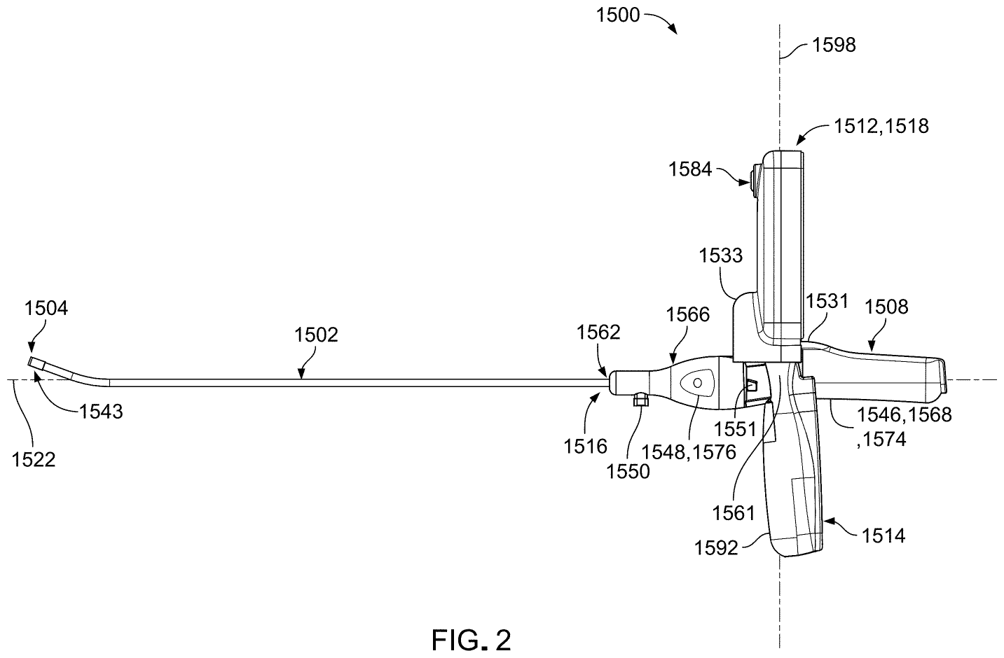

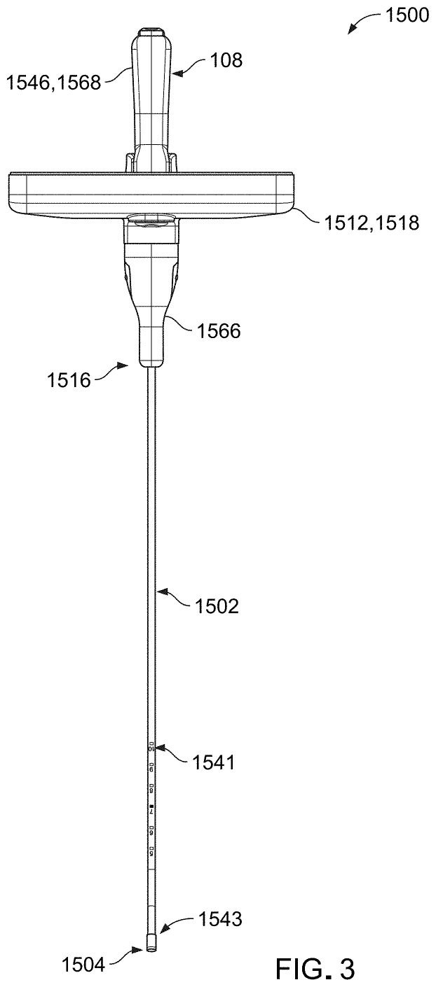

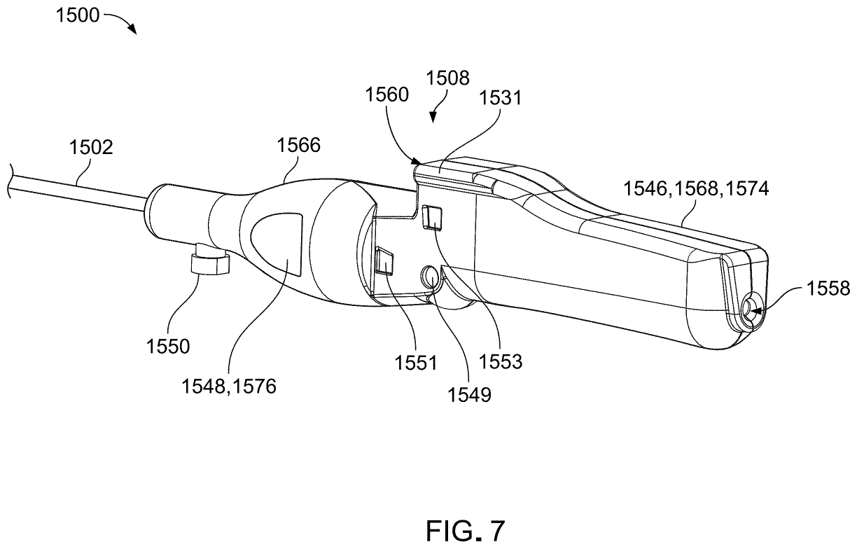

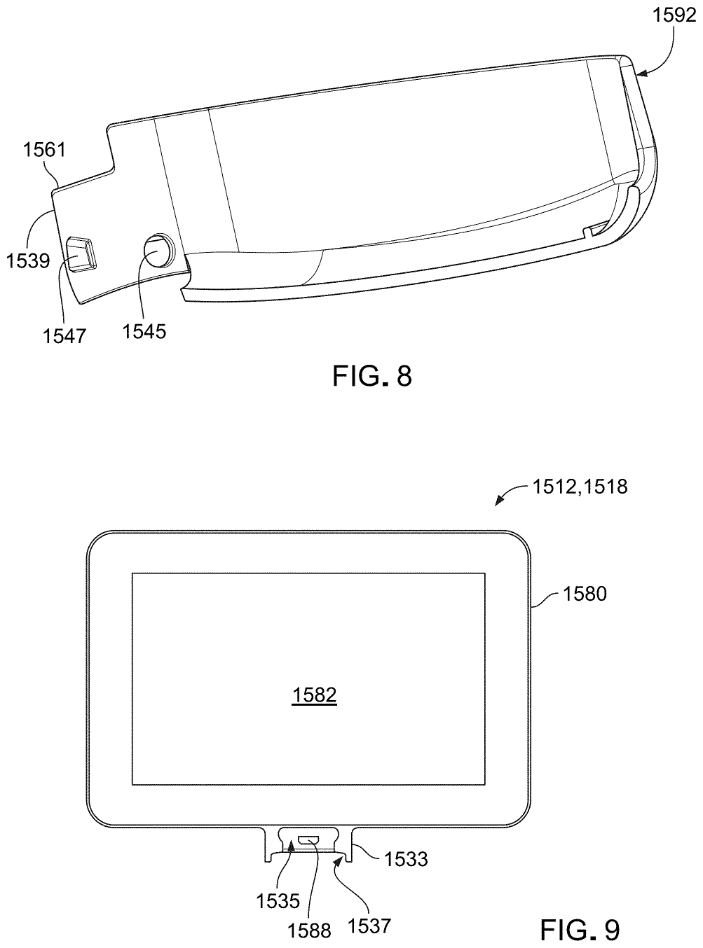

A proximal portion 1568 of the housing 1546 provides a grip 1574 that can be used to manipulate the endoscopic device 1500, and the handle 1514 is pivotable with respect to the proximal portion 1568. Referring particularly to FIG. 8, the handle 1514 defines a circular protrusion 1545 by which the handle 1514 can rotate with respect to the proximal portion 1568 and a polygonal protrusion 1547 by which a position of the handle 1514 can be locked with respect to the proximal portion 1568. Referring particularly to FIG. 7, the proximal portion 1568 of the housing 1546 defines a circular recess 1549 that is sized to receive the circular protrusion 1545 to allow the handle 1514 to pivot with respect to the proximal portion 1568. The proximal portion 1568 further defines a polygonal recess 1551 by which the handle 1514 can be locked in an in-line configuration (e.g., a “pencil-grip” configuration) and a polygonal recess 1553 by which the handle 1514 can be locked in an off-axis configuration (e.g., a “pistol grip” configuration in which the handle 1514 is oriented antiparallel to the connection hub 1508), as illustrated in FIGS. 2 and 3, and as will be discussed in more detail below.

The housing 1546 of the connection hub 1508 typically has a length (e.g., as measured along the primary axis 1522 of the cannula 1502) of about 10 cm to about 20 cm (e.g., about 15 cm) and a maximum width of about 20 cm to about 30 cm (e.g., about 25 cm). The proximal portion 1568 of the housing 1546 (e.g., excluding the grip 1574) typically has a width of about 1.4 cm to about 1.8 cm (e.g., about 1.6 cm). The housing 1546 is typically made of one or more materials, such as ABS, polycarbonate, and copolyester, and is typically manufactured via injection molding.

The imaging system 1504 includes the camera 1542, LEDs 1538 located on opposite sides of the camera 1542 to evenly illuminate surrounding tissues for image acquisition, the camera actuator 1548, one or more electrical cables (e.g., one or more video and control cables, not shown) that extend from the camera and the LEDs to the camera actuator 1548 and to the connection port 1560, and other electrical components that provide electrical communication amongst the various components of the imaging system 1504 and the connection port 1560.

In some embodiments, the one or more electrical cables extend through the lumen 1528 of the cannula 1502. In some embodiments, the one or more electrical cables extend within channels in a sidewall of the cannula 1502. In some embodiments, the imaging system 1504 includes a flex circuit member to carry the electrical communications instead of one or more electrical cables. The push buttons 1576 are flexible components that may be formed from an overmolded elastomer such that when either or both of the push buttons 1576 are depressed, the push buttons 1576 temporarily move internal components of the camera actuator to 1548 to initiate image capture.

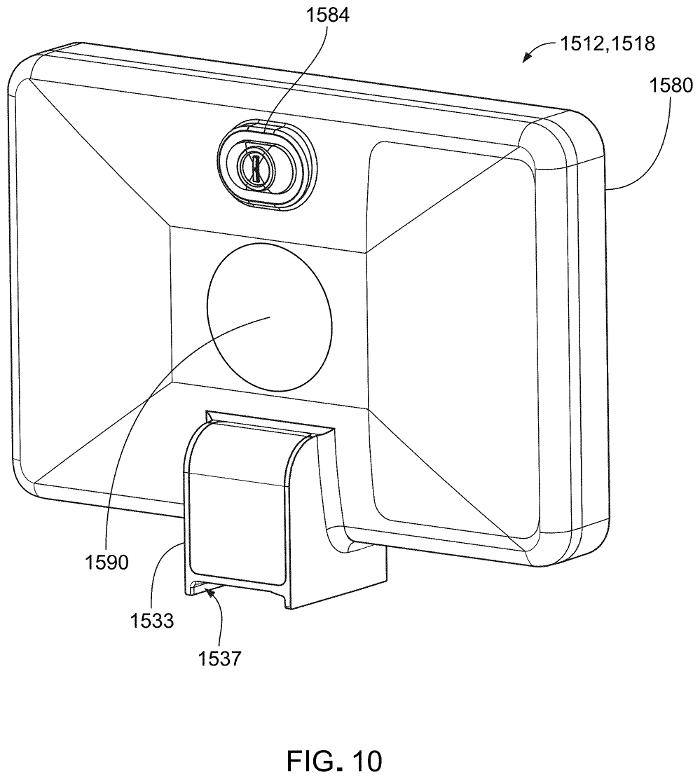

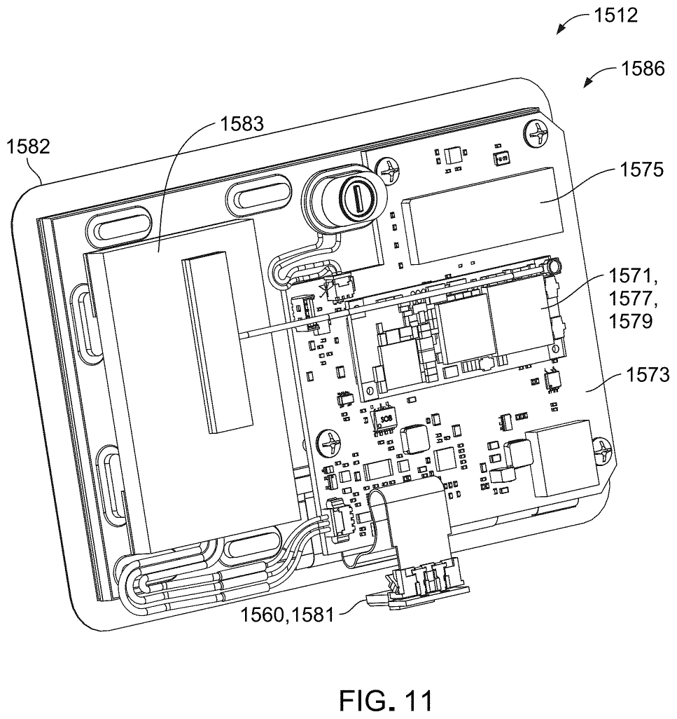

Referring to FIGS. 9-11, the display 1512 includes a housing 1580, a screen 1582, a power button 1584 located along an upper rear surface of the display 1512, internal electronics 1586, an electrical connector 1588 (e.g., a micro HDMI connector or another type of connector) that mates with the connection port 1560 of the connection hub 1508 to relay signals between the imaging system 1504 and the internal electronics 1586, and a round metal plate 1590. The metal plate 1590 is designed to be supported by or otherwise interface with a magnet and/or a flexible accessory arm when the display 1512 is decoupled from the single-use portion 1516 of the endoscopic device 1500.

The display 1512 further includes an attachment piece 1533 that defines a slot 1535 at which the display 1512 can be slid proximally to be attached to the connection hub 1508 along the flanges 1531 of the connection port 1560 and at which the display 1512 can be slid distally from the flanges 1531 to disassemble the display 1512 from the connection hub 1508. The slot 1535 typically has a maximum width of about 10 cm to about 30 cm (e.g., about 20 cm) for proper frictional mating with the flanges 1531 of the connection port 1560. The attachment piece 1533 further defines opposite channels 1537 that are complementary to and that contact edges 1539 of the handle 1514 when the display 1512 is secured to the connection hub 1508.

The housing 1580 of the display 1512 typically has a length of about 11 cm to about 15 cm (e.g., about 13 cm), a width of about 7 cm to about 9 cm (e.g., about 8 cm), and a height of about 2 cm to about 4 cm (e.g., about 3 cm). Referring particularly to FIG. 3, the display 1512 is typically oriented at an angle of about 80° to about 100° (e.g., about 90°) with respect to the connection hub 1508, as measured between the primary axis 122 of the cannula 1502 and a central axis 1598 of the display 1512. The housing 1580 of the display 1512 is typically manufactured via injection molding. The display 1512 typically has a weight of about 0.2 kg to about 0.3 kg.

The internal electronics 1586 are programmed or otherwise configured to process or manipulate data acquired by the camera, to generate GUIs displayed on the screen 1582, to transmit data via a wired connection between the display 1512 and the imaging system 1504, to transmit data wirelessly between the display 1512 and other devices (e.g., a computer, a smart phone, or a tablet) that are not mechanically connected to the endoscopic device 1500, to power the endoscopic device on and off, and to implement various user-selected settings of the endoscopic device 1500. The internal electronics 1586 include a microprocessor 1571, a printed circuit board (PCB) 1573, an internet service provider (ISP) 1575, a WiFi module 1577, a battery management circuit, a current monitor circuit, an on board memory 1579 (e.g., non-volatile storage memory), a universal serial bus (USB) interface 1581, and a rechargeable battery 1583 with a charging capacity of about 1400 mAh needed to carry out the functionality of the imaging system 1504 and other features of the endoscopic device 1500.

The electrical connecter (omitted from the figures for clarity) serves multiple purposes, including video-out to an external display, connector to an AC adapter for charging the rechargeable battery, and/or as a port to a host PC for downloading and uploading images, video and/or settings, as well as for charging the rechargeable battery. The on board memory is used to accept flash memory cards used to store images, video and/or settings for the endoscopic device 1500.

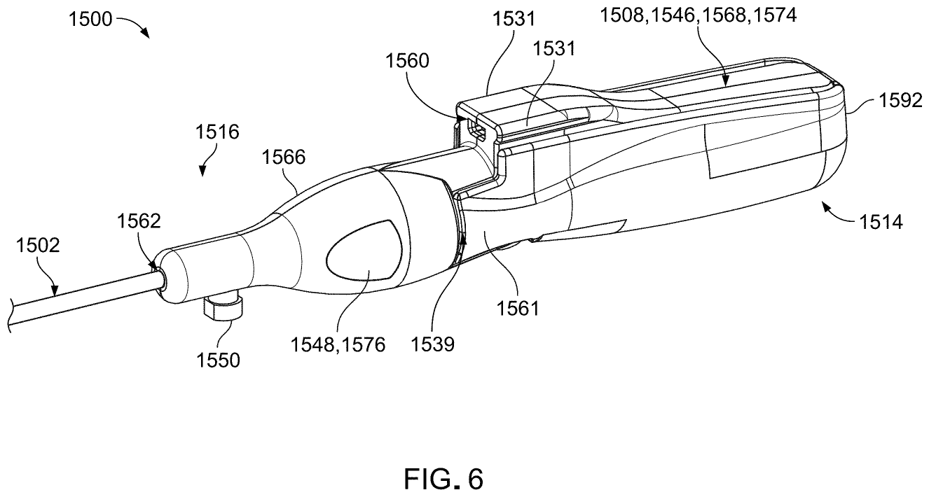

Referring to FIGS. 2 and 6, the handle 1514 defines a gripping portion 1592 by which the handle 1514 can be grasped to be pivoted towards the connection hub 1508 to an in-line configuration (shown in FIG. 6) in which the handle 1514 is oriented and stowed in-line with the connection hub 1508. The gripping portion 1592 defines a channel 1596 that surrounds the proximal portion 1568 of the connection hub 1508 when the handle 1514 is oriented in the in-line configuration. In addition to the gripping portion 1592, the handle 1514 also defines two opposite tabs 1561 that define the protrusions 1547 that snap into the recesses 1551 disposed along the proximal portion 1568 of the connection hub 1508 to maintain the handle 1514 in the in-line configuration. The tabs 1561 include respective protrusions 1565 that prevent the display 1512 from being slid along the connection port 1560 to be attached to the connection hub 1508 when the handle 1514 is oriented in the in-line configuration (e.g., the protrusions 1565 provide an obstruction to movement of the display 1512). Accordingly, the tabs 1561 prevent the display 1512 from being attached to the reusable portion 1516 of the endoscopic device 1500 in a configuration in which the display 1512 may not be stably balanced on the connection hub 1508 and in which a user's hand (e.g., grasping the handle 1514 would obstruct a view of the display screen 1582.

The handle 1514 can also be pivoted from the in-line configuration to an off-axis configuration (shown in FIG. 6) in which the handle 1514 is oriented an angle of about 90° to about 100° (e.g., about 95°) with respect to the connection hub 1508 (shown in FIG. 2) to provide a pistol-type grip. When the handle 1514 is in the off-axis configuration, the display 1512 can be slid along the connection port 1560 to be attached to the connection hub 1508. The channels 1537 of the attachment piece 1533 are in contact with the edges 1539 along the tabs 1561 of the handle 1514 when the display 1512 is attached to the connection hub 1508.

To adjust the handle 1514 between the in-line configuration and the off-axis configuration, the force applied to the gripping portion 1592 of the handle 1514 must be high enough to push the protrusions 1547 of the tabs 1561 out of the recesses 1551 or the recesses 1553 along the connection hub 1508. The handle 1514 is accordingly made of one or more materials (e.g., including polycarbonate, copolyester, and ABS) that allow the tabs 1561 to flex with respect to the recesses 1551, 1553, as well as that can chemically withstand various sterilization solutions and procedures. The handle 1514 has a length of about 7 cm to about 12 cm (e.g., about 9 cm) and a width of about 1 cm to about 3 cm (e.g., about 2 cm). The single-use portion 1516 of the endoscopic device 1500 (e.g., including the cannula 1502, the imaging system 1504, the connection hub 1508, and the handle 1514) typically has a weight of about 0.2 kg to about 0.4 kg.

The display 1512 can be attached to the connection hub 1508 prior to inserting the cannula 1502 into the patient, the display 1512 can be unattached to (e.g., and in wireless communication with) the connection hub 1508 while the cannula 1502 is inserted into the patient (e.g., with the handle 1514 in the in-line configuration), or the display 1512 can be connected to the connection hub 1508 at the connection port 1560 by a display cable prior to inserting the cannula 1502 into the patient (e.g., with the handle 1514 in the in-line configuration). Referring again to FIG. 2, to attach the display 1512 to the connection hub 1508, the display 1512 is placed near the proximal portion 1568 and moved proximally to slide the slot 1535 onto the flanges 1531 of the connection hub 1508 until the electrical connector 1588 mates with the connection port 1560. The display 1512 is held in place on the flanges 1531 by a frictional fit. To disconnect the display 1512 from the connection hub 1508, the display 1512 is pulled distally relative to the connection hub 1560 to move the slot 1535 off of the flanges 1531.

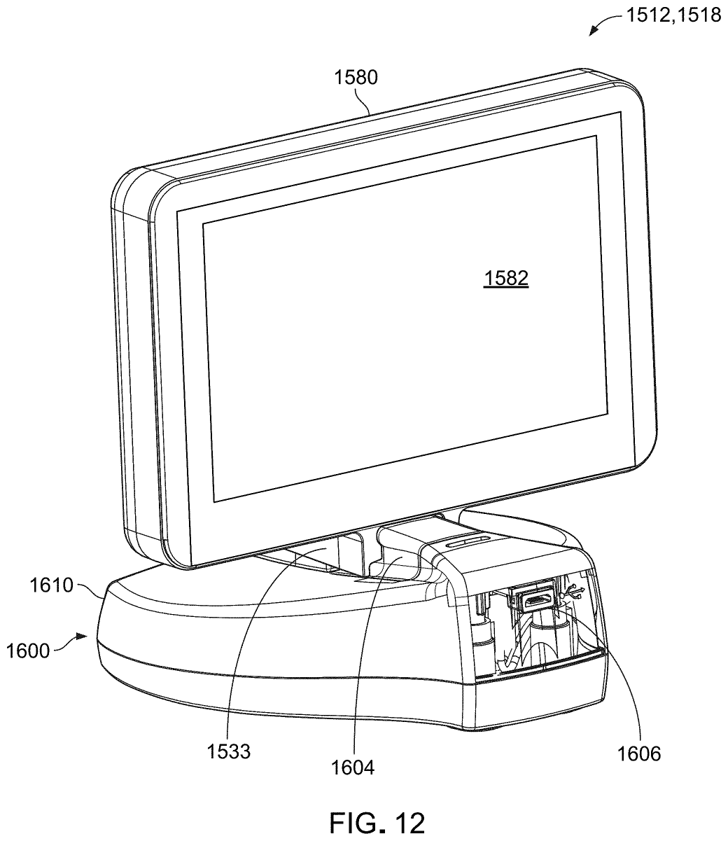

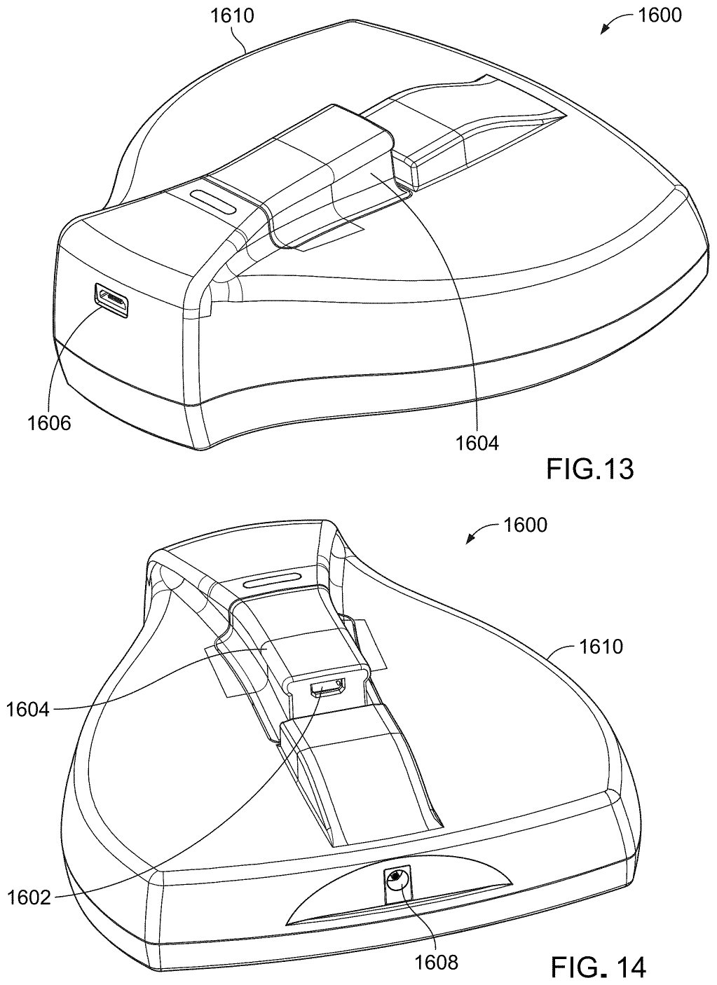

FIGS. 12-14 illustrate a docking station 1600 to which the display 1512 of the endoscopic device 1500 can be mounted for charging and data transfer. The docking station 1600 includes a connection port 1602 (e.g., a micro HDMI port) that can be connected to the electrical connector 1588 of the display 1512, a mount 1604 that guides proper positioning of the display 1512 on the docking station 1600 (e.g., the attachment piece 1533 of the display 1512 can be slid along the mount 1604 towards the connection port 1602), a connection port 1606 to which a cable can be connected to transfer data from the display 1512 to another electronic or computing device, a power connector 1608 to which a power cable can be connected to the docking station 1600, and a housing 1610 that encloses internal electronics. The docking station 1600 typically has a length of about 9 cm to about 13 cm (e.g., about 11 cm), a width of about 9 cm to about 13 cm (e.g., about 11 cm), and a total height of about 3 cm to about 5 cm (e.g., about 4 cm). Example materials from which the housing 1610 may be made include ABS, polycarbonate, and copolyester. The docking station 1600 typically has a weight in a range of about 0.15 kg to about 0.25 kg.

As discussed above, the display 1512 may supported by or otherwise interfaced at the metal plate 1590 with an accessory component when the display 1512 is decoupled from the single-use portion 1516 of the endoscopic device 1500. Example accessory components include a rigid or flexible arm designed to attach to the display 1512 and a cable permitting the display 1512 to be positioned separately from the single-use portion 1516 of the endoscopic device 1500 while remaining functionally connected to the single-use portion 1516.

An endoscopic device may be substantially similar in construction and function in several aspects to the endoscopic device 1500 discussed above, but can include an alternative cannula instead of the cannula 1502. In some embodiments, the cannula may have two configurations such that the cannula is steerable. For example, the cannula may have a straight configuration or a bent configuration. Such steerable configurations can allow a user (e.g., a clinician) to manipulate a distal end of the cannula to observe intrauterine anatomy without manipulating the entire cannula body to achieve visualization of a desired area.

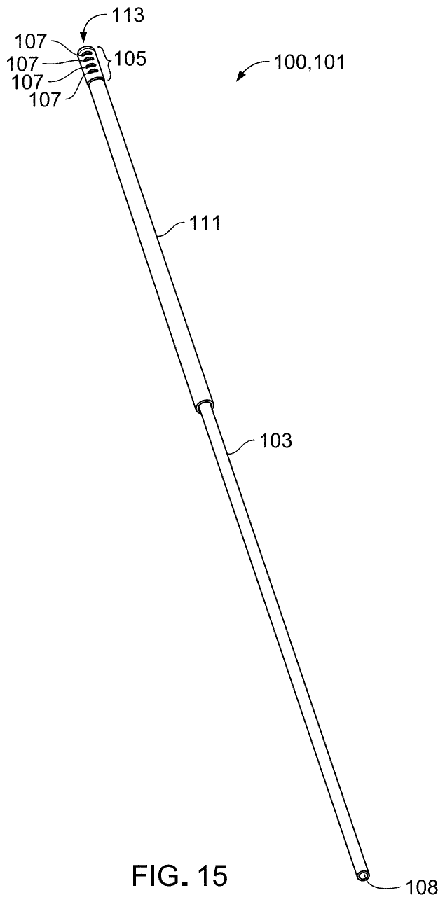

FIGS. 15-25 illustrate examples of cannulas 101, 201, 301, 401, and 501 that are elongate, generally tubular members that can have a straight configuration or a bent configuration and are sized to pass through a cervix and into a uterus of a patient. The cannulas 101, 201, 301, 401, 501 are respectively part of endoscopic devices 100, 200, 300, 400, 500 that otherwise include the connection hub 1508 or a similar connection hub, the handle 1514 or a similar handle, the display 1512 or a similar display, and an imaging system. The imaging system is substantially similar in construction and function to the imaging system 1504 and accordingly includes the camera 1542 and one or more of the LEDs, except that a positioning of the camera 1542 and any LEDs along the distal tip may be different. In some embodiments the cannulas 101, 201, 301, 401, and 501 are adjustable between the straight configuration and the bent configuration. The shafts 103, 202, 302, 402, and 502 define a lumen (e.g., the lumen 108 of cannula 101) that houses one or more electrical cables of the imaging system 1504 and that allows for passage of fluids between the distal tip 113 and the connection hub 1408. In some embodiments, the lumen allows for passage of a working tool extending distally from connection hub 1408.

Cannulas 101, 201, 301, 401, and 501 include elongate shafts 103, 202, 302, 402, and 502, respectively. That is, cannula 101 includes an elongate shaft 103, cannula 201 includes an elongate shaft 202, cannula 301 includes an elongate shaft 302, cannula 401 includes an elongate shaft 402, and cannula 501 includes an elongate shaft 502. Elongate shafts 103, 202, 302, 402, and 502 include a distal end portion sized for insertion in a body cavity (e.g., a vaginal cavity or a uterus). Cannulas 103, 202, 302, 402, and 502 include the camera 1542 and one or more LEDs 1538 (shown in FIG. 4) of the imaging system and are secured to the distal end portion of elongate shafts 103, 202, 302, 402, and 502. The camera 1542 can be used for viewing the body cavity (e.g., a vaginal cavity or a uterus). The distal end portion can be adjustable between a straight configuration and a bent configuration.

Referring to FIGS. 15-20, each of cannulas 101, 201, and 301 include a sheath 111 or a sheath 310. The sheaths 111 and 310 define an interior region for receiving the elongate shafts 103, 202, and 302. The sheaths 111 and 310 coaxially surround the elongate shafts 103, 202, and 302. The sheaths 111 and 310 are slidable along the elongate shafts 103, 202, and 302 to adjust the elongate shafts 103, 202, and 302 between the straight configuration and the bent configuration. For example, the sheath can be used to uncurl the distal end portion when in a bent configuration. Sheaths 111 and 310 can be further used to direct the camera 1542 disposed at the distal tip 113, 213, 313 to a desired location within a uterus of a patient.

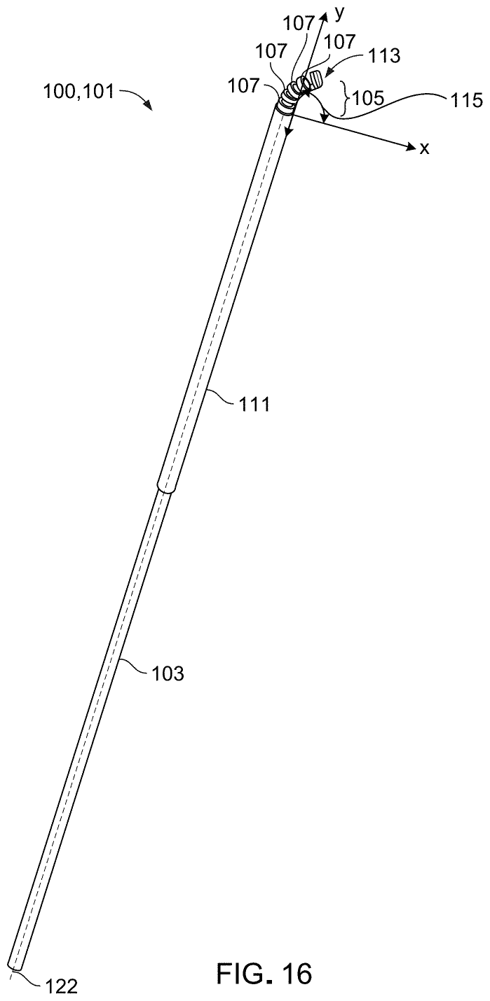

Referring particularly to FIGS. 15 and 16, cannula 101 includes an elongate shaft 103 having a distal end portion 105. The distal end portion 105 of the elongate shaft 103 can be adjustable between a straight configuration, as shown in FIG. 15, and a bent configuration, as shown in FIG. 16. The distal end portion 105 of the elongate shaft 103 includes one or more relief cuts 107 that generate the bent configuration shown in FIG. 16. For example, the one or more relief cuts 107 cause the distal end portion 105 to bend or curl. The one or more relief cuts 107 typically have an axial width of about 0.5 to about 2.0 mm. The distal portion 105 typically has a diameter of about 3.0 to about 0.5 mm, and the elongate shaft 103 typically has an outer diameter of about 2.5 to about 4.5 mm. In some embodiments, elongate shaft 103 includes a total of 4 to 8 relief cuts 107. In some embodiments, the one or more relief cuts 107 may be embodied as slits, notches, or kerfs made by a cutting tool in order to facilitate bending of the elongate shaft 103. In some embodiments, the elongate shaft 103 is made of a flexible material.

In some embodiments, the bent configuration caused by the one or more relief cuts 107 may be the natural or trained shape of the distal end portion 105 of the elongate shaft 103. In other words, no force may be required to be applied to the distal end portion 105 in order to generate the bent configuration. In other examples, the elongate shaft 103 can be made of a flexible material whose natural or trained shape is curled. In some embodiments, a natural or trained shape of the distal end portion 105 of the elongate shaft 103 provides the bent configuration. In some embodiments, the distal end portion 105 of the elongate shaft 103 can curve at an angle 115 of about 1 degree to about 45 degrees when in the bent configuration.

The sheath 111 is slidable along the elongate shaft 103 to adjust the elongate shaft 103 between the straight configuration and the bent configuration. In some embodiments, sheath 111 is a rigid sheath. In some embodiments, sheath 111 is a semi-rigid sheath. Example materials from which the sheath 111 is typically made include, but are not limited to polycarbonate, polypropylene, and acrylonitrile butadiene styrene (ABS). In some embodiments, an endoscopic device that is otherwise similar to the endoscopic device 100 may include a shaft that does not allow passage of an operative instrument. The lumen 108 of shaft 103 typically has a diameter of about 3.0 to about 5.0 mm.

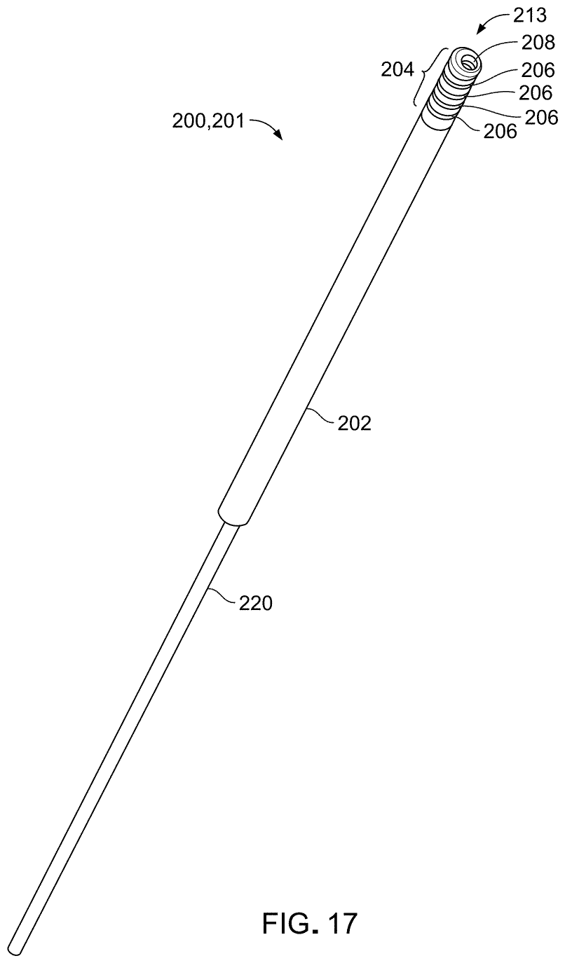

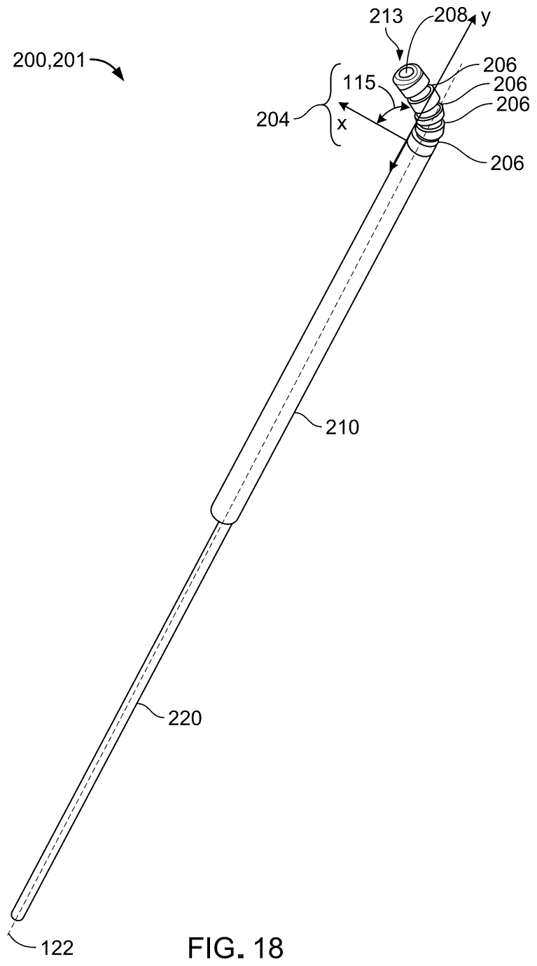

Referring particularly to FIGS. 17 and 18, cannula 201 includes an elongate shaft 202 having a distal end portion 204 and the sheath 111 (illustrated in FIGS. 15 and 16). The distal end portion 204 of the elongate shaft 202 can be adjustable between a straight configuration, as shown in FIG. 17, and a bent configuration, as shown in FIG. 18. The distal end portion 204 of the elongate shaft 202 includes one or more relief cuts 206 that generate the bent configuration shown in FIG. 18. For example, the one or more relief cuts 206 cause the distal end portion 204 to bend or curl. The one or more relief cuts 206 typically have an axial width of about 0.5 to about 2.0 mm. The distal end portion 204 typically has a diameter of about 3.0 to about 5.0 mm, and the elongate shaft 202 typically has an outer diameter of about 2.5 to about 5.0 mm. In some embodiments, elongate shaft 202 includes a total of 4 to 8 relief cuts 206. In some embodiments, the one or more relief cuts 206 may be embodied as slits, notches, or kerfs made by a cutting tool in order to facilitate bending of the elongate shaft 202.

In some embodiments, the bent configuration caused by the one or more relief cuts 206 may be the natural or trained shape of the distal end portion 204 of the elongate shaft 202. In other words, no force may be required to be applied to the distal end portion 204 in order to generate the bent configuration. In other examples, the elongate shaft 202 can be made of a flexible material whose natural or trained shape is curled. In some embodiments, a natural or trained shape of the distal end portion 204 of the elongate shaft 202 provides the bent configuration. In some embodiments, the distal end portion 204 of the elongate shaft 202 can curve at an angle 115 of about 1 degree to about 45 degrees when in the bent configuration. The sheath 111 (shown in FIGS. 15 and 16) is slidable along the elongate shaft 202 to adjust the elongate shaft 202 between the straight configuration and the bent configuration.

In some embodiments, the shaft 202 defines a lumen 208 that is configured to allow passage of an operative instrument 220 from a proximal end of the elongate shaft 202 through the distal end portion 204 of the elongate shaft 202. In some embodiments, the shaft 202 is a semi-rigid shaft. In some embodiments, the operative instrument 220 provides additional stability during steering of the distal end portion 204. The lumen 208 of shaft 202 typically has a diameter of about 3.0 to about 5.0 mm. In some embodiments, lumen 208 houses one or more electrical cables of the imaging system 1504 and allows for passage of fluids between the distal tip 313 and the connection hub 1508.

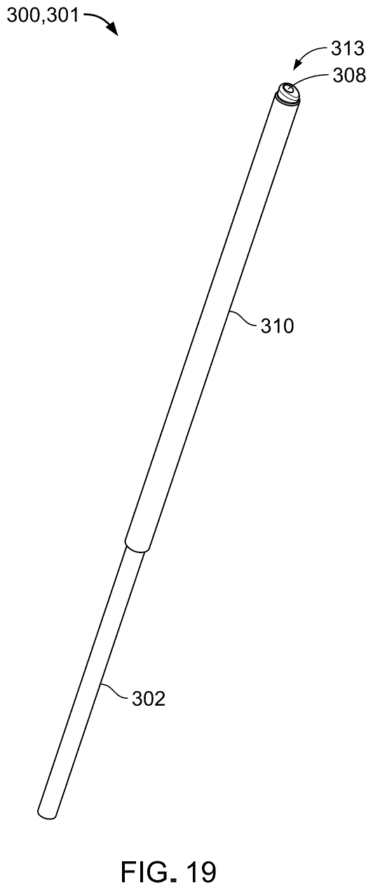

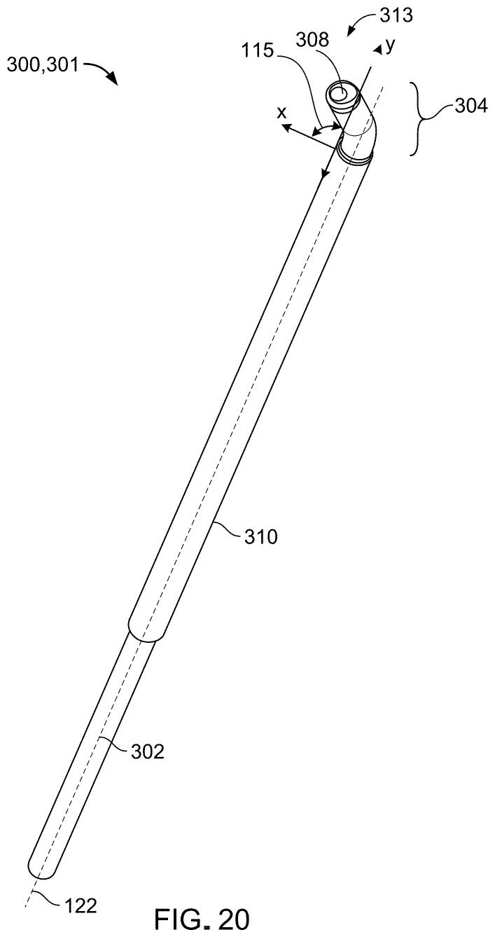

Referring particularly to FIGS. 19 and 20, cannula 301 includes an elongate shaft 302 having a distal end portion 304. The distal end portion 304 of the elongate shaft 302 can be adjustable between a straight configuration, as shown in FIG. 19, and a bent configuration, as shown in FIG. 20. The elongate shaft 302 defines a lumen 308. The lumen 308 of elongate shaft 308 typically has a diameter of about 3.0 to about 5.0 mm. In some embodiments, lumen 308 is configured to allow passage of an operative instrument from a proximal end of the elongate shaft 402 through the distal end portion 404 of the elongate shaft 402. In some embodiments, lumen 308 houses one or more electrical cables of the imaging system 1504 and allows for passage of fluids between the distal tip 313 and the connection hub 1508. In some embodiments, lumen 308 allows for passage of a working tool extending distally from connection hub 1508.

The elongate shaft 302 is made from a memory alloy material that can be trained to “remember” the bent configuration. The distal end portion 304 of the elongate shaft 302 can transition between the straight and bent configurations in response to a temperature change of the memory alloy material. An example material from which the elongate shaft 302 is typically made includes, but is not limited to nickel-titanium alloy (nitinol). Other non-limiting example materials include copper-aluminum-nickel, iron-manganese-silicon (FE—Mn—Si), and copper-zinc-aluminum (Cu—Zn—Al).

In some embodiments, the elongate shaft 302 has a one-way shape-memory effect. A memory alloy material exhibits a one-way shape-memory effect when its original shape is changed to a second shape (e.g., a bent or stretched shape) in a cold state and holds this second shape until heated above a transition temperature. Upon heating, the second shape changes back to the original shape and once the memory alloy material cools again, the material retains the original shape. For example, the distal end portion 304 of elongate shaft 302 has a bent configuration in a cold state at a first temperature (e.g., room temperature or about 25 degrees Celsius) and can be straightened into the straight configuration in the cold state. Then, upon an increase in temperature above a transition temperature (e.g., a second temperature), the straight configuration can change back to the bent configuration, which is retained upon cooling from the second temperature to the first temperature. Alternatively, in some embodiments, the distal end portion 304 of elongate shaft 302 has a straight configuration in a cold state at a first temperature (e.g., room temperature or about 25 degrees Celsius) and can be bent into the bent configuration in the cold state. Then, upon an increase in temperature above a transition temperature (e.g., a second temperature), the bent configuration can change back to the straight configuration, which is retained upon cooling from the second temperature to the first temperature.

In some embodiments, the elongate shaft 302 has a two-way shape-memory effect. A memory alloy material exhibits a two-way shape-memory effect when the memory alloy material “remembers” two different shapes: one at a relatively low temperature and one at a relatively high temperature. For example, the distal end portion 304 of elongate shaft 302 having a two-way shape-memory effect can have a straight configuration in a cold state at a first temperature (e.g., room temperature or about 25 degrees Celsius). Then, upon an increase in temperature above a transition temperature (e.g., a second temperature), the straight configuration can change to a pre-determined bent configuration. Finally, upon a decrease in temperature (i.e., from the second temperature to the first temperature), the bent configuration can change into the original, straight configuration. Thus, both configurations can be “remembered” at both the first and second temperatures by the memory alloy material. Alternatively, the elongate shaft 302 can exhibit the same two-way shape-memory effect but instead can have a bent configuration in the cold state and change to a straight configuration in a hot state, above a transition temperature. The first temperature typically ranges from about −190 to about 200° C. The second temperature typically ranges from about −190 to about 200° C. The transition temperature can typically range from about −190 to about 200° C.

In some embodiments, the distal end portion 304 of the elongate shaft 302 can have a bent configuration in the shape of a pig tail, a hook, a spiral, a curve, a loop, or a coil. In some embodiments, the distal end portion 304 of the elongate shaft 302 can curve at an angle 115 of about 1 degree to about 45 degrees when in the bent configuration.

In some embodiments, the distal end portion 304 of the elongate shaft 302 can be trained for memory in more than one shape. For example, the distal end portion 304 of the elongate shaft 302 can have a straight configuration, a first bent configuration, and a second bent configuration. Furthermore, the user (e.g., the clinician) can select any configuration by varying the temperature of the alloy. For example, the user may expose the elongate shaft 302 to a first temperature to select the first bent configuration. Similarly, the user may expose the elongate shaft 302 to a second temperature to select the second bent configuration. The cannula 301 may further include a heating element and/or a cooling element, a temperature sensor, and a temperature display (omitted from the figures) that enables the user to control the temperature of the elongate shaft 302 thereby controlling the desired shaft configuration. Once in a bent configuration, sheath 310 can be slid over the distal end portion 304 of the elongate shaft 302 to control angle 115 thereby controlling the direction of the camera 1542 (shown in FIG. 4) disposed at the distal end portion 304. Sheath 301 can be slid distally or proximally, with respect to the user, as needed, in order to adjust angle 115.

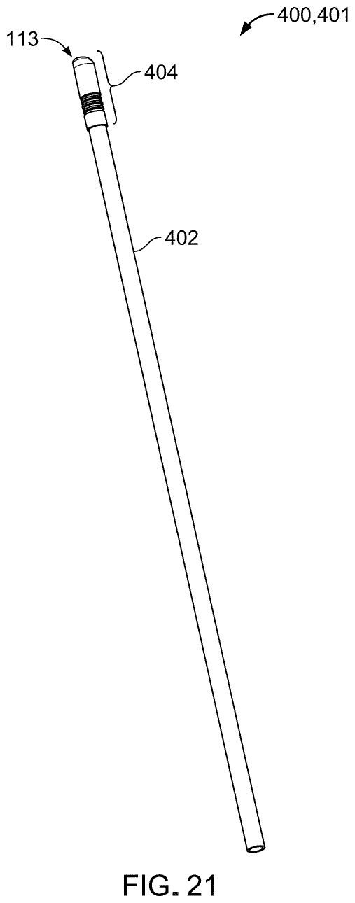

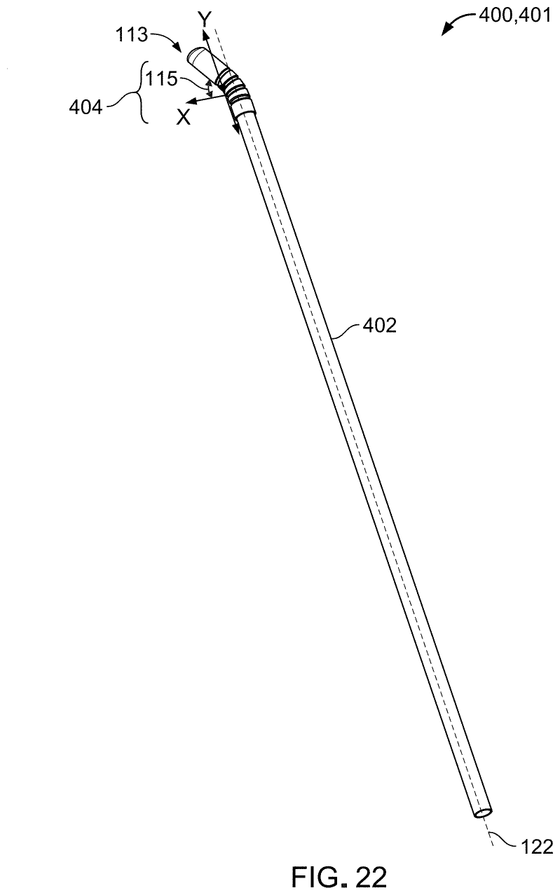

Referring particularly to FIGS. 21 and 22, cannula 401 includes an elongate shaft 402 having a distal end portion 404. The distal end portion 404 of the elongate shaft 402 can be adjustable between a straight configuration, as shown in FIG. 21, and a bent configuration, as shown in FIG. 22. The distal end portion 404 of the elongate shaft 402 is made of a bendable plastic that generates the bent configuration shown in FIG. 22. A user can create a desired shape or bend by manipulating or bending the distal end portion 404 of the elongate shaft 402. The user can manipulate or bend the distal end portion 404 of the elongate shaft 402 to direct the camera 1542 (shown in FIG. 4) disposed at the distal end portion 404 for better visualization of intrauterine anatomy. For example, the user can manipulate or bend the distal end portion 404 prior to insertion into a body cavity (e.g., a uterus of a patient), retrieve and adjust the angle 115, and re-insert the cannula 401 if needed to obtain a better image when viewing the body cavity. In some embodiments, the distal end portion 404 of the elongate shaft 402 can curve at an angle 115 of about 1 degree to about 45 degrees with respect to the x- and y-axes, when in the bent configuration. Example bendable plastics from which the distal end portion 404 of the elongate shaft 402 is typically made include, but are not limited to polypropylene and ABS. In some embodiments, the elongate shaft 402 is made of a bendable plastic.

In some embodiments, the shaft 402 defines a lumen that is configured to allow passage of an operative instrument from a proximal end of the elongate shaft 402 through the distal end portion 404 of the elongate shaft 402. In some embodiments, the shaft 402 is a semi-rigid shaft. In some embodiments, the shaft 402 provides stability during steering of the distal end portion 404. The lumen of shaft 402 typically has a diameter of about 3.0 to about 5.0 mm.

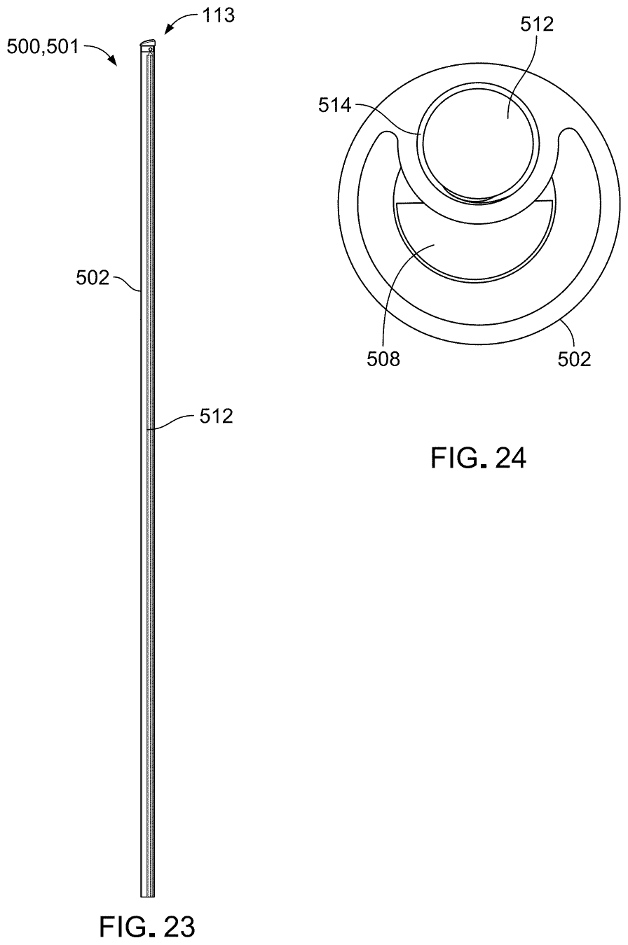

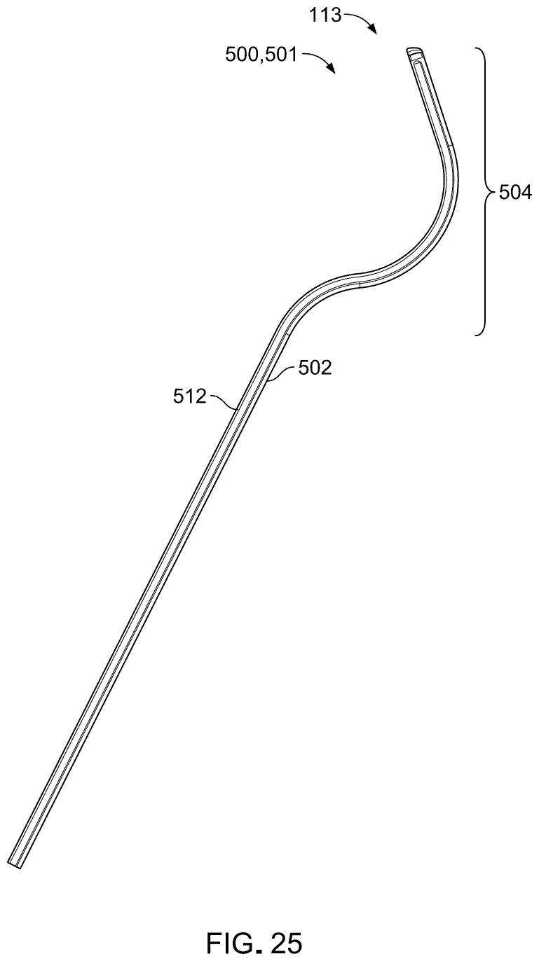

Referring particularly to FIGS. 23, 24, and 25, cannula 501 includes an elongate shaft 502 having a distal end portion 504. The distal end portion 504 of the elongate shaft 502 can be adjustable between a straight configuration, as shown in FIG. 23, and a bent configuration, as shown in FIG. 25. The shaft 502 defines a first lumen 508 having a crescent shape and a second lumen 514 having a circular cross-sectional shape, as shown in FIG. 24. The first lumen 508 can allow passage of an operative instrument from a proximal end of the elongate shaft 402 through the distal end portion 404 of the elongate shaft 402, can house one or more electrical cables of the imaging system 1504, and can allows for passage of fluids between the distal tip 113 and the connection hub. The first lumen 508 of shaft 502 typically has a crescent-shaped cross-sectional area of about 8 to about 12 mm². The first lumen 508 of shaft 502 typically has a crescent-shaped cross-sectional area sized about 60% to about 80% of a circular cross-sectional area of the second lumen 514.

Cannula 501 further includes a malleable metal rod 512 disposed within the second lumen 514 defined by the elongate shaft 502. The second lumen 514 of shaft 504 typically has a circular-shaped cross-sectional area of about 1.0 to about 2.5 mm. The second lumen 514 of shaft 502 typically has a circular-shaped cross-sectional area sized about 40% to about 20% of the crescent-shaped cross-sectional area of the first lumen 508. The malleable metal rod 512 is configured to adjust the distal end portion 504 of the elongate shaft 502 into the straight configuration or the bent configuration. The malleable metal rod 512 typically has a diameter of about 1.0 to about 3.0. Example metals from which the malleable metal rod 512 typically made include, but are not limited to stainless steel.

A user can create a desired shape or bend by manipulating bending the distal end portion 504 of the elongate shaft 502. For example, FIG. 25 shows the distal end portion 504 of the elongate shaft 502 having a hook shape in the bent configuration. In some embodiments, the distal end portion 504 of the elongate shaft 502 can have a bent configuration in the shape of a pig tail, a spiral, a curve, a loop, or a coil. The number of configurations and shapes of the distal end portion 504 of the elongate shaft 502 are not limited to the aforementioned shapes as the user can manipulate or bend the elongate shaft 502. The user can manipulate the distal end portion 504 of the elongate shaft 502 to direct the camera 1542 disposed at the distal end portion 504 for better visualization of intrauterine anatomy. For example, the user can bend the distal end portion 504 prior to insertion into a body cavity (e.g., a uterus of a patient), retrieve and adjust the shape or bend, and re-insert the cannula 501 if needed to obtain a better image when viewing the body cavity.

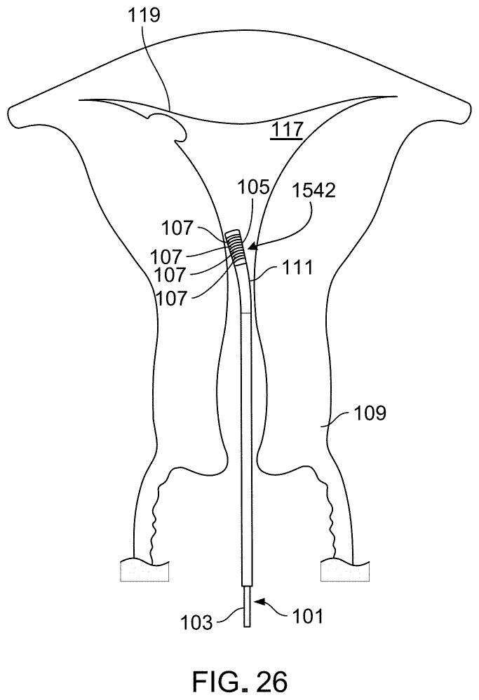

FIG. 26 illustrates the cannula 201 of the endoscopic device 200 within a uterine cavity 117 of a patient during an endoscopic procedure (e.g., a hysteroscopic procedure). A clinician uses the handle 1514 (shown in FIGS. 1-3) to insert an elongate shaft 103 of the endoscopic cannula 101 in a straight configuration into a body cavity (e.g., a cervix 109) of a patient. Therefore, the sheath 111 coaxially surrounds the distal end portion 105 of the endoscopic cannula 101 when the endoscopic cannula 101 is inserted into the cervix 109 of a patient. Furthermore, sheath 111 remains accessible to the user (e.g., a clinician) outside of the uterine cavity while the endoscopic cannula 101 is within a uterine cavity 117 of the patient. The clinician advances the endoscopic cannula 101 distally until the distal end portion 105 of the endoscopic cannula 101 is positioned at a desired location and at a desired orientation within a uterine cavity 117 of the patient. The clinician adjusts the distal end portion 105 of the elongate shaft 103 from a straight configuration to a bent configuration by sliding sheath 111 proximally along the elongate shaft 103 of the endoscopic cannula 101. Thus, the bent configuration is provided with a natural or trained shape (e.g., a curved shape) at the distal end portion 105 of the elongate shaft 103.

The clinician may further adjust the position and/or orientation the distal end portion 105 of the endoscopic cannula 101 by sliding the sheath 111 proximally or distally as needed along the elongate shaft 103. The clinician may acquire an image of the body cavity (e.g., the cervix 109) of the patient by using the camera 1542 secured to the distal end portion 105 of the elongate shaft 103. Once the clinician is ready to withdraw the endoscopic cannula 101, the clinician adjusts the configuration of the distal end portion 105 of the endoscopic cannula 101 from a bent configuration to a straight configuration by sliding sheath 111 distally along the elongate shaft 103. Next, the clinician proceeds to withdraw the cannula 101 in the straight configuration.

Alternatively, in some embodiments, the clinician may change the configuration of the distal end portion 105 of the endoscopic cannula 101 from a straight configuration to a bent configuration prior to insertion into the cervix 109 of the patient. For example, the clinician may bend the distal end portion 105 of the elongate shaft 103 at one or more relief cuts 107 of the elongate shaft 103.

In some cases (as in the example of FIGS. 26-30), the clinician views an abnormality 119, such as an endometrial lesion, a uterine fibroid (e.g., a myoma), a uterine polyp, a cancerous tumor, an adhesion, a hyperplastic growth (or, in some cases, another anatomical feature of interest, such as a healthy-appearing tissue disposed near a region of interest) within the uterine cavity 117 via a video stream or via one or more images captured by the endoscopic cannula 101 and displayed on a monitor of the handset. Upon viewing the abnormality 119, the clinician may decide to perform an operation (e.g., a biopsy procedure, a polypectomy, an excision, or a cautery) within the uterine cavity 117 to further examine or to treat the abnormality 119.

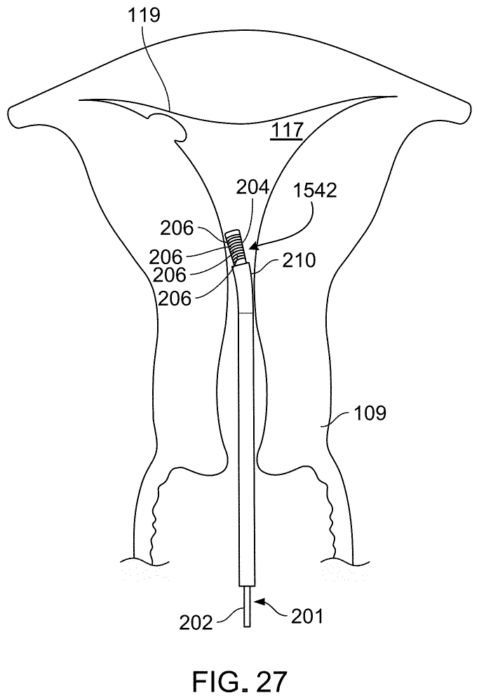

FIG. 27 illustrates the cannula 201 of the endoscopic device 200 within a uterine cavity 117 of a patient. The clinician can adjust the configuration of the distal end portion 204 of the endoscopic cannula 201 from a straight configuration to a bent configuration by sliding sheath 210 proximally along the elongate shaft 202 of the endoscopic cannula 201. The clinician may further adjust the position and/or orientation the distal end portion 204 of the endoscopic cannula 201 by sliding the sheath 210 proximally or distally as needed along the elongate shaft 202. The clinician may further insert a working tool (e.g., an operative instrument) into a proximal end of the elongate shaft 202, through a lumen of the elongate shaft 202, and through the distal end portion 204 of the elongate shaft 202. The clinician may manipulate the operative instrument to perform a surgery in the body cavity (e.g., the cervix 109) of a patient.

Once the clinician is ready to withdraw the endoscopic cannula 201, the clinician adjusts the configuration of the distal end portion 204 of the endoscopic cannula 201 from a bent configuration to a straight configuration by sliding sheath 210 distally along the elongate shaft 202. Alternatively, in some embodiments, the clinician may change the configuration of the distal end portion 204 of the endoscopic cannula 201 from a straight configuration to a bent configuration prior to insertion into the cervix 109 of the patient.

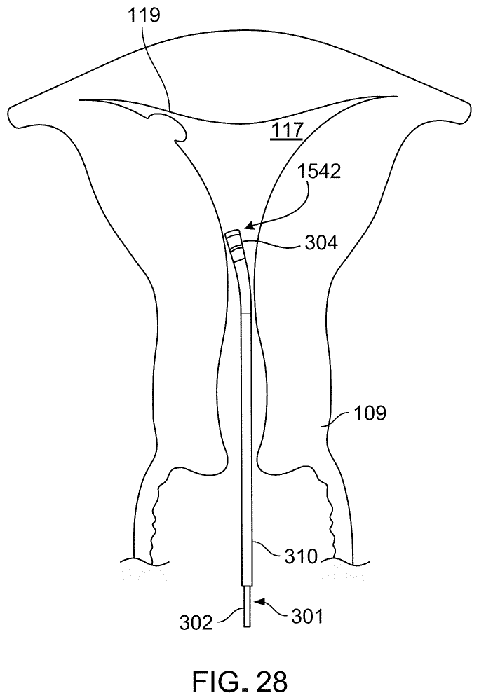

FIG. 28 illustrates the cannula 301 of the endoscopic device 300 within a uterine cavity 117 of a patient. The clinician can use the handle of the endoscopic device 300, attached to the endoscopic cannula 301, to insert the endoscopic cannula 301 made from a memory alloy material into a cervix 109 of a patient. The clinician may change the configuration of the distal end portion 304 of the endoscopic cannula 301 from a straight configuration to a bent configuration by changing the temperature of the elongate shaft 302 from a first temperature (e.g., room temperature or about 25 degrees Celsius) to a second temperature (e.g., body temperature or about 37 degrees Celsius). The clinician may further adjust the orientation and/or position of the distal end portion 304 of the endoscopic cannula 301 in a bent configuration by changing the temperature of the elongate shaft 302 from a second temperature (e.g., body temperature or about 37 degrees Celsius) to a third temperature.

Once the clinician is ready to withdraw the endoscopic cannula 301, the clinician changes the temperature of the endoscopic cannula 301 from the second or third temperatures to the first temperature thereby changing the configuration of the distal end portion 304 of the endoscopic cannula 301 from a bent configuration to a straight configuration. Next, the clinician proceeds to withdraw the cannula 301 in the straight configuration. Alternatively, in some embodiments, the clinician may change the configuration of the distal end portion 304 of the endoscopic cannula 301 from a straight configuration to a bent configuration prior to insertion into the cervix 109 of the patient.

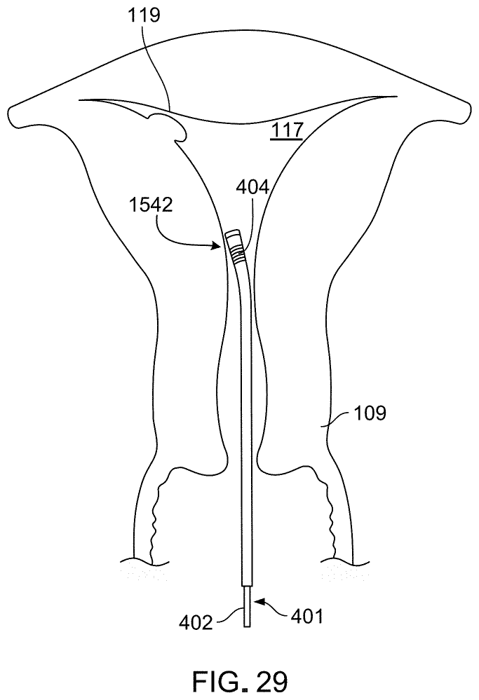

Referring to FIG. 29, illustrates the cannula 401 of the endoscopic device 400 within a uterine cavity 117 of a patient. The clinician can adjust the configuration of the distal end portion 404 of the endoscopic cannula 401 made from a bendable plastic from a straight configuration to a bent configuration prior to insertion into the cervix 109 of the patient. The clinician bends or manipulates the bendable distal end portion 404 of the elongate shaft 402 to create a desired shape and/or angle and proceeds to insert the endoscopic cannula 401 into the cervix 109 of the patient.

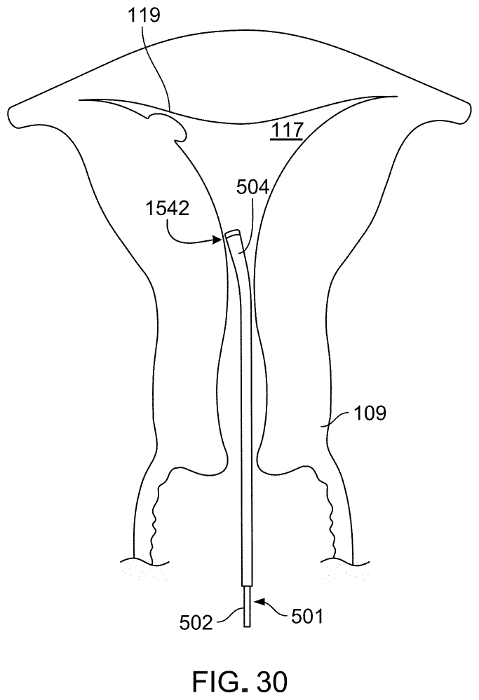

FIG. 30 illustrates the cannula 501 of the endoscopic device 500 within a uterine cavity 117 of a patient. The clinician can adjust the configuration of the distal end portion 504 of the endoscopic cannula 501, encasing a malleable metal rod, from a straight configuration to a bent configuration prior to insertion into the cervix 109 of the patient. The clinician bends or manipulates the bendable distal end portion 504 of the elongate shaft 502 to create a desired shape and/or angle and proceeds to insert the endoscopic cannula 501 into the cervix 109 of the patient.

While the above-discussed endoscopic devices and cannulas have been described and illustrated as including certain dimensions, shapes, arrangements, configurations, and material formulations, and with respect to certain methods, in some embodiments, an endoscopic device or a cannula that is similar in construction and function to any of the above-discussed endoscopic devices or cannulas may include one or more dimensions, shapes, arrangements, configurations, and/or materials formulations that are different from the ones discussed above or may be used with respect to methods that are modified as compared to the methods described above. Other embodiments are within the scope of the following claims.

What is claimed is:

1. An endoscopic cannula, comprising:
an elongate shaft having a distal end portion sized for insertion in a body cavity; and
a camera secured to the distal end portion of the elongate shaft for viewing the body cavity;
wherein the distal end portion of the elongate shaft is adjustable between a straight configuration and a bent configuration to examine the body cavity.

2. The endoscopic cannula of claim 1, wherein the distal end portion of the elongate shaft comprises one or more relief cuts that generate the bent configuration.

3. The endoscopic cannula of claim 1, wherein the elongate shaft is made of a flexible material.

4. The endoscopic cannula of claim 3, wherein a natural or trained shape of the distal end portion of the elongate shaft provides the bent configuration.

5. The endoscopic cannula of claim 3, further comprising a malleable metal rod disposed within the elongate shaft and configured to adjust the distal end portion of the elongate shaft into the straight configuration or the bent configuration.

6. The endoscopic cannula of claim 1, wherein a lumen of the elongate shaft is configured to allow passage of an operative instrument from a proximal end of the elongate shaft through the distal end portion of the elongate shaft.

7. The endoscopic cannula of claim 1, wherein the elongate shaft is made of a memory alloy material.

8. The endoscopic cannula of claim 1, further comprising a rigid sheath defining an interior region for receiving the elongate shaft.

9. The endoscopic cannula of claim 8, wherein the rigid sheath is slidable along the elongate shaft to adjust the elongate shaft between the straight configuration and the bent configuration.

10. The endoscopic cannula of claim 8, wherein the distal end portion of the elongate shaft transitions between the straight and bent configurations in response to a temperature change of the memory alloy material.

11. An endoscope, comprising:
an elongate shaft having a distal end portion sized for insertion in a body cavity;
a camera secured to the distal end portion of the elongate shaft for viewing the body cavity; and
a handle extending from a proximal end of the elongate shaft wherein the distal end portion of the elongate shaft is adjustable between a straight configuration and a bent configuration to examine the body cavity.

12. A method of using an endoscope, the method comprising:
inserting an elongate shaft of the endoscope into a body cavity of a patient;
adjusting a distal end portion of the elongate shaft from a straight configuration to a bent configuration; and
acquiring an image of the body cavity using a camera secured to the distal end portion of the elongate shaft.

13. The method of claim 12, further comprising bending the distal end portion of the elongate shaft at one or more relief cuts of the elongate shaft.

14. The method of claim 12, wherein the elongate shaft is made of a flexible material.

15. The method of claim 14, further comprising providing the bent configuration with a natural or trained shape at the distal end portion of the elongate shaft.

16. The method of claim 15, further comprising manipulating a malleable metal rod within a lumen of the elongate shaft to adjust the distal end portion of the elongate shaft into the straight configuration or the bent configuration.

17. The method of claim 12, further comprising inserting an operative instrument into a proximal end of the elongate shaft, through a lumen, and through the distal end portion of the elongate shaft.

18. The method of claim 17, further comprising manipulating the operative instrument to perform a surgery in the body cavity.

19. The method of claim 12, wherein the elongate shaft is made of a memory alloy material.

20. The method of claim 19, further comprising sliding a rigid sheath over the elongate shaft to adjust the distal end portion of the elongate shaft into the straight configuration or the bent configuration.

21. The method of claim 19, further comprising changing a temperature of the memory alloy material to adjust the distal end portion of the elongate shaft into the straight configuration or the bent configuration.
